(19) United States
(12) Patent Application Publication
(10) Pub. No.: US 2021/0393430 A1
(43) Pub. Date: Dec. 23, 2021

(54) Knee Brace

(71) Applicant: Ottobock SE & Co. KGaA, Duderstadt (DE)

(72) Inventors: Matthias Schilling, Weissenborn-Lüderode (DE); Gordon Siewert, Göttingen (DE); Alexander Schmitt, Kassel (DE)

(73) Assignee: Ottobock SE & Co. KGaA, Duderstadt (DE)

(21) Appl. No.: 17/290,097

(22) PCT Filed: Oct. 14, 2019
(86) PCT No.: PCT/EP2019/077819

§ 371 Date: Apr. 29, 2021

(30) Foreign Application Priority Data
Oct. 29, 2018 (DE) 10 2018 126 990.0

(51) Int. Cl. A61F 5/01

(52) U.S. Cl. CPC A61F 5/0123; A61F 2005/0179; A61F 2005/0158

(57) ABSTRACT

The invention relates to a knee orthosis with a lower leg part that extends in a lower leg direction, and an upper leg part that extends in a lower leg direction, and at least one locking device, wherein the lower leg part and the upper leg part are connected to each other by at least one swivel joint such that they can be swiveled, the lower leg part and/or the upper leg part comprise a lateral rail and a medial rail, and the lower leg direction encloses a knee frontal angle with the upper leg direction, wherein the knee orthosis has a first accommodation element and a second accommodation element which can be fixed in different positions with different knee frontal angles relative to one another by means of the at least one locking device.

The invention relates to a knee orthosis with a lower leg part that extends in a lower leg direction and an upper leg part that extends in an upper leg direction and at least one locking device, wherein the lower leg part and the upper leg part are connected by at least one swivel joint such that they can be swivelled, the lower leg part and/or the upper leg part comprises a lateral rail and a medial rail, and the lower leg direction forms a knee frontal angle with the upper leg direction.

Knee orthoses in a wide range of embodiments are known from the prior art and used, for example, to treat osteoarthritis in the knee. In addition, it can be used to treat knee deformities, especially “genu varum” and “genu valgum”. These deformities differ in the knee frontal angle of the leg, that is, the angle between the longitudinal direction of the upper leg and the longitudinal direction of the lower leg, which is in the frontal plane. An orthosis for these applications also has a knee frontal angle between the upper leg direction and the lower leg direction. This knee frontal angle also lies in the frontal plane.

A knee orthosis according to the preamble is described, for instance, in U.S. Pat. No. 6,981,957 B2. Such orthoses should be able to be used for “genu varum” deformities as well as for “genu valgum”, i.e. “O” and “X” legs. To this end, it has been proven to be advantageous if an angle between the lower leg part and the upper leg part is designed to be adjustable. The lower leg part is used for attaching to the patient's lower leg, while the upper leg part is attached to the upper leg. Since the wearer is a patient with a knee deformity, the lower leg and upper leg are not parallel but at an angle to each other, which must be replicated by the knee orthosis. So as not to have to manufacture a separate orthosis for each angle or to provide a large number of different knee orthoses, it is known from the prior art to design this angle to be adjustable. In the case of an orthosis according to the preamble from the prior art, this is achieved by the fact that the lateral rail and the medial rail are designed to be variable in length and have a frontal joint. While the swivel joint, with which the upper leg part and the lower leg part are connected to each other, allows swivelling about the knee axis and thus a natural movement of the knee, the frontal joints of the prior art have a swivel axis that extends perpendicular to this direction. In this way, the fact that the length of the medial rail and the lateral rail is adjustable allows an angle between the upper leg part and the lower leg part to be adjusted and adapted to the personal needs of the wearer. The disadvantage is that the device from the prior art is structurally complex and therefore expensive.

WO 2008/104149 A1 discloses a knee orthosis in which the lower leg part has two proximal sides that can be swivelled relative to one another about a swivel axis and fixed in different orientations relative to one another. There is no change in the position of the two sides relative to each other.

The invention aims to further develop a knee orthosis in such a way that it is structurally simple to produce and yet can still be used for a wide variety of knee deformities.

The invention solves the problem by way of a knee orthosis according to the preamble of claim 1, which is characterized in that the knee orthosis has a first accommodation element and a second accommodation element which can be fixed in different positions with different knee frontal angles relative to one another by means of the at least one locking device.

In the different positions in which the first accommodation element and the second accommodation element can be fixed to each other, the knee orthosis has different knee frontal angles. Therefore, the angle between the lower leg direction and the upper leg direction changes depending on the position. The lower leg direction is preferably the direction in which the lower leg of the wearer of the knee orthosis extends when the knee orthosis is worn as intended. Accordingly, the upper leg direction is preferably the direction in which the upper leg of the wearer of the knee orthosis extends when the knee orthosis is worn as intended.

In order to move from a position in which the first accommodation element and the second accommodation element are or can be attached to each other into a second position that is different to the first, it is not sufficient to swivel the two accommodation elements relative to each other. This would not change the position of the two accommodation elements relative to each other; rather, it would only change the orientation of the two elements relative to each other.

Preferably, the lower leg part and the upper leg part are connected to each other via two swivel joints such that they can be swivelled. One of the swivel joints is preferably arranged laterally and one medially. The two swivel axes of the two swivel joints are preferably arranged coaxially. The different knee frontal angles are preferably created by changing a ratio of the distances between the contact area where the two accommodation elements abut or are attached to each other and the two joints connecting the lower leg part to the upper leg part.

The accommodation elements may be designed, for example, in the form of half-shells or shell-shaped elements and, when attached to one another, completely enclose the patient's upper or lower leg. In contrast to the prior art, which at this point provides for connecting rods between the medial rail and the lateral rail that permanently connect the two rails to each other, the invention provides for the first accommodation element and the second accommodation element to be provided between the lateral rail and the medial rail, which are or can be connected to one another such that they can be detached. They can be fixed to each other via the locking device. This means that the locking device must be released in order to change the position of the lateral accommodation element relative to the medial accommodation element.

It is advantageous if the various positions in which the accommodation elements can be attached relative to each other are infinitely adjustable. Alternatively, various discrete, i.e. spaced-apart, positions are provided in which the two accommodation elements are positioned relative to one another and can be fixed by the locking device. In different positions, the preferably fixed connection of the lateral accommodation element to the lateral rail and the medial accommodation element to the medial rail results in a change in the angle between the lower leg part and the upper leg part, meaning that different knee deformations can be accommodated.

In a preferred configuration, the first accommodation element is attached to the lateral rail and the second accommodation element to the medial rail. Preferably, the first accommodation element is only attached to the lateral rail and not the medial rail, and the second accommodation element only to the medial rail and not the lateral rail.

The first accommodation element and the second accommodation element are preferably attached to the lateral rail and the medial rail respectively. Preferably, the two accommodation elements are arranged respectively on the two rails such that they can be swivelled. In addition, however, the accommodation elements also remain attached to each other, wherein this may occur in different positions that result in the different knee frontal angles described above.

The first accommodation element and the second accommodation element preferably each comprise at least one positive-locking element, which are designed to correspond with one another. As a result, it is possible to connect and fix the two accommodation elements relative to each other and on each other in a particularly simple manner.

In a preferred configuration, at least one of the positive-locking elements is a slot, for example a curved elongated hole, while the other positive-locking element is mounted in this slot such that it can be moved, the slot preferably being designed in such a way that an orientation of the first accommodation element relative to the second accommodation element remains constant. The other positive-locking element can be a pin or a bolt, for example, which protrudes from the respective accommodation element and through the slot. The positive-locking element in the form of the pin is mounted in the slot such that it can be displaced, thereby allowing various positions of the two accommodation elements relative to each other to be realized. In an especially preferred configuration, the slot is designed in the shape of an elongated hole that runs in the shape of an arc.

If the slot is designed in such a way that an orientation of the two accommodation elements relative to each other, i.e. an angle between the two, remains constant, this ensures that the angle between the areas of the lower leg part and/or the upper leg part at which the two accommodation elements are arranged also remains constant. These are preferably medial rails and lateral rails. If these remain parallel regardless of the position in which the two accommodation elements are arranged in relation to each other, a fit, once set, is maintained, as is the associated comfort when wearing the orthosis. Such a movement of the two accommodation elements is known as collinear movement. As such, the angle preferably also remains constant when the knee frontal angle changes.

In this case, it is advantageous if the locking device is a screw connection. The other positive-locking element, which is mounted in the slot such that it can be displaced, can be equipped with an outer thread, for example, on which a nut can be placed. When the nut is tightened, the accommodation element with the slot is clamped between the other accommodation element and the nut, causing it to be fixed and locked in place. Of course, different washers or intermediate elements can be provided here in order to distribute the occurring pressure loads as optimally as possible.

Preferably, the first accommodation element and/or the second accommodation element comprises multiple positive-locking elements arranged at an offset to each other.

The at least one locking device is preferably configured to fix the first accommodation element to the second accommodation element or vice-versa. This can, but does not have to, occur via the previously described positive-locking elements.

Preferably, the width of the upper leg part of the knee orthosis is adjustable. For this purpose, multiple slots are provided that preferably run parallel to each other. The positive-locking element that can be displaced in the slot is guided, for example, through a hole that is arranged in the accommodation element that does not comprise the slot. The positive-locking element can be guided in the various slots, which changes the width of the upper leg part. Alternatively or additionally, multiple holes may be provided in the respective accommodation element through which the positive-locking element can be guided.

In a preferred configuration, the locking device is designed in the form of a velcro element and at least one velcro counter-element. It is advantageous if the velcro element is arranged on one of the accommodation elements, i.e. the first or second accommodation element, while the velcro counter-element is preferably arranged on the respective other accommodation element. A locking element designed in this way can be provided as an addition or an alternative to the previously described positive-locking elements. It is thus possible, for example, to equip the accommodation elements with the previously described positive-locking elements and, in addition, to configure a locking device with velcro elements and velcro counter-elements.

The lateral rail and/or the medial rail preferably have at least two components that are connected to each other by at least one frontal joint. A frontal joint features a swivel axis that extends perpendicular to the swivel axis of the swivel joint and thus preferably also perpendicular to the axis of the knee joint. Here, a frontal joint is called a frontal joint because its swivel axis extends perpendicular to a frontal plane; as a result, the two components that are connected by the frontal joint can be swivelled against each other in a frontal plane. For example, the frontal joints are designed in the form of hinges, for instance in the form of film hinges.

Due to the fact that the at least two components of the lateral rail and/or the medial rail can be swivelled, the medial rail and the lateral rail can accommodate the different positions in which the two accommodation elements can be fixed relative to one another and follow any desired movement of the components relative to one another.

The locking device is preferably configured to fix the frontal joint. By fixing at least one frontal joint, but preferably both frontal joints, a further displacement and a change of the position of the lateral accommodation element relative to the medial accommodation element is no longer possible, so that a locking device designed in this way also fixes the two elements relative to each other without the locking device having to be arranged on one or both of the accommodation elements for this purpose. Locking devices which block a movement of a joint have been known from the prior art for many years.

It is advantageous if the frontal joint, preferably every frontal joint, features an adjustment device with which an angular position of the frontal joint can be adjusted. In particular, a reproducible and easily adjustable angular position can be achieved in this way.

Alternatively or additionally, the medial rail and/or the lateral rail is designed to be elastic. For example, it can be made from a metal, especially a spring steel, and pre-tensioned into a different position by displacing the two accommodation elements relative to each other. This also allows an angle between the lower leg part and the upper leg part to be adjusted to the respective physical conditions and also a force that can be exerted on the knee or on the upper leg and the lower leg.

The medial rail and the lateral are preferably designed such that they are not variable in length.

At least one attachment element is arranged on the upper leg part and/or the lower leg part, preferably at least one in each case, by means of which the knee orthosis can be attached to the upper leg and/or the lower leg of the wearer. Preferably, they are straps that are equipped with corresponding closing elements. The straps are preferably attached to the respective component via tabs. Other attachment elements, for example belts, bands or cable systems, such as the cable system arranged by BOA Technology Inc. can be used additionally or alternatively. The cable system from BOA Technology Inc. has the advantage that, due to the easy adjustability of the length of the actual cable element, the orthosis can be easily mounted and individually adjusted.

In the following, an example of an embodiment of the present invention will be explained in more detail by way of the attached figures: They show:

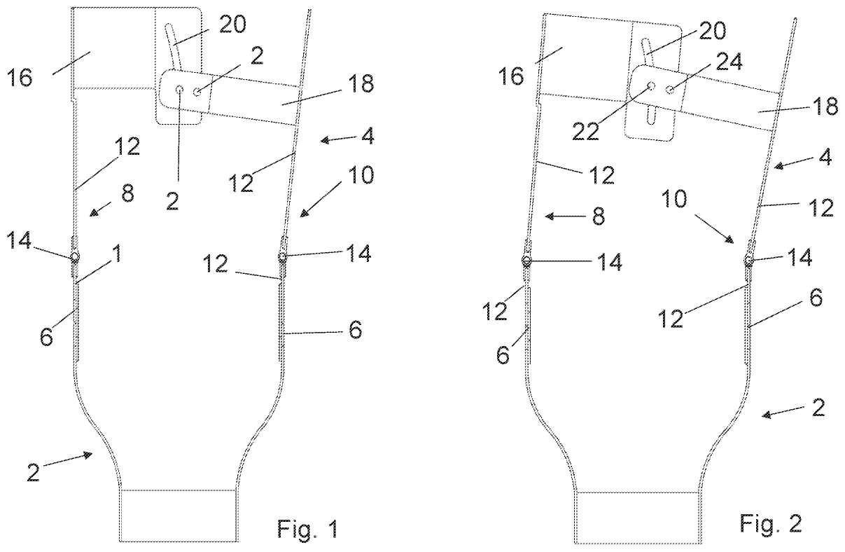

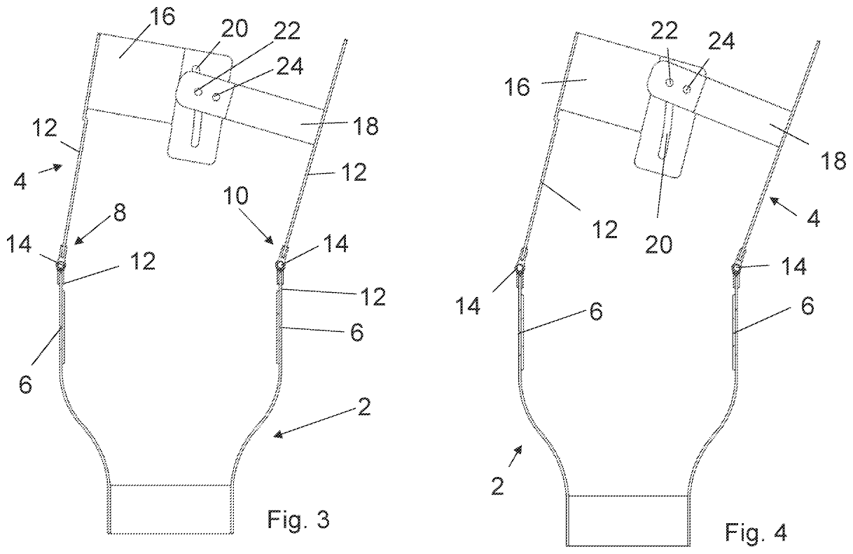

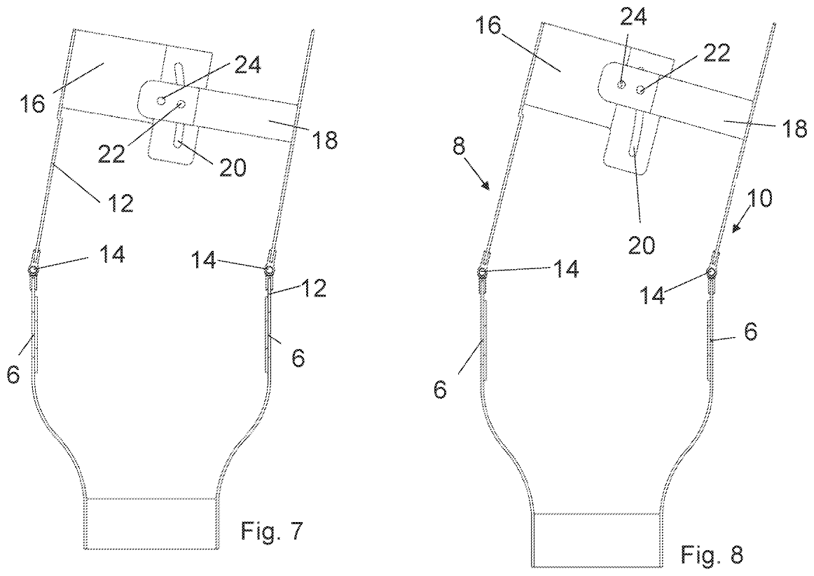

FIGS. 1 to 8 a knee orthosis according to an example of an embodiment of the present invention in various positions,

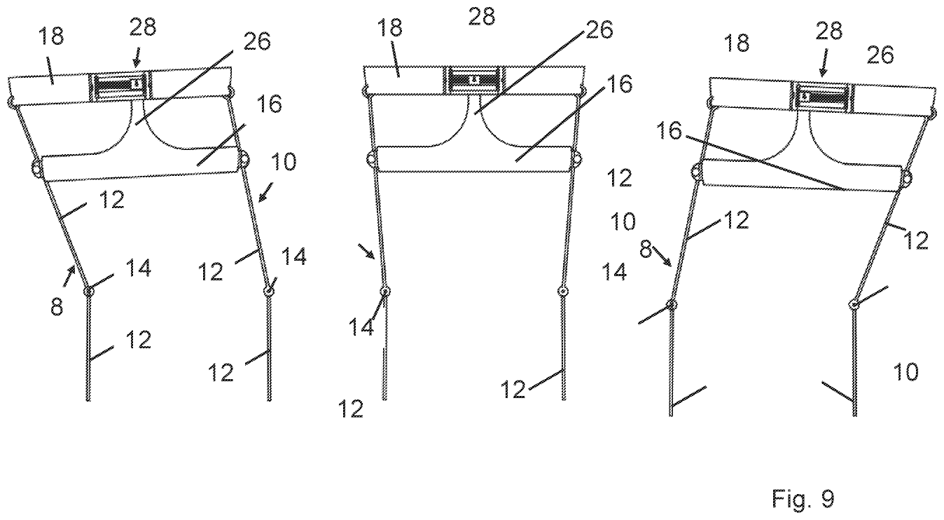

FIGS. 9 to 11 a knee orthosis according to a further example of an embodiment of the present invention.

The knee orthosis depicted in FIGS. 1 to 8 has a lower leg part 2 and an upper leg part 4 which are connected to each other by two swivel joints 6 in the example of an embodiment shown. When the knee orthosis is mounted, the lower leg part 2 is arranged on the lower leg of the wearer and the upper leg part 4 on the upper leg of the wearer. The swivel joints 6 allow a swivelling of the lower leg part 2 relative to the upper leg part 4 and are preferably aligned in such a way that the swivel axis of the two swivel joints 6 coincides with the knee axis of the wearer.

In the example of an embodiment shown, the upper leg part 4 comprises a lateral rail 8 and a medial rail 10, each of which has two components 12 in the example of an embodiment shown that are connected to each other by a front joint 14 such that they can be swivelled.

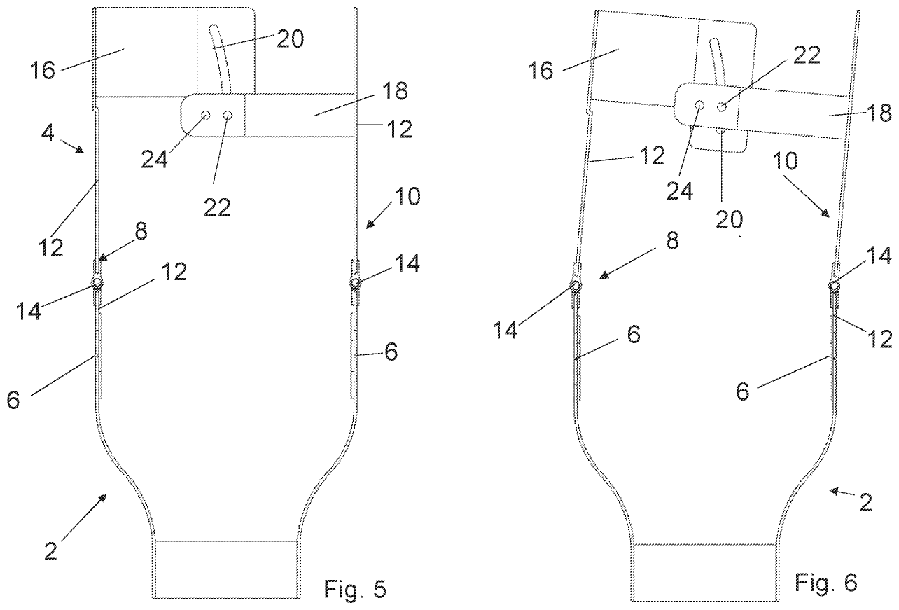

The lateral rail 8 also features a first accommodation element 16 and the medial rail 10 a second accommodation element 18. The first accommodation element 16 is arranged on the lateral rail 8 and features a positive-locking element in the form of a slot 20, in which a second positive-locking element in the form of a pin 22, which is arranged on the second accommodation element 18, is arranged such that it can be displaced. The second accommodation element 18 is arranged on the medial rail 10. The pin 22 is arranged in one of two available holes 24 in the example of an embodiment shown and guided through the slot 20. It is therefore possible to design a width of the upper leg part 4 to be adjustable. FIGS. 5 to 8 depict the knee orthosis, wherein the pin 22 is now arranged in the second of the two holes 24. Of course, more than two holes 24 may be provided to make the width of the upper leg part 4 adjustable in several steps. Additionally or alternatively, multiple slots 22 may be provided, which preferably run parallel to one another. This also renders the width of the upper leg part 4 adjustable.

FIGS. 1 to 4 depict the same knee orthosis, wherein the pin 22 is arranged in different positions in the slot 20. This causes the two frontal joints 14 to swivel, so that an angle between the upper leg part 4 and the lower leg part 2 changes. The first accommodation element 16 can be fixed to the second accommodation element 18, for example, via a locking device, not depicted here. Alternatively or additionally, a locking device may be be provided that prevents a further swivelling of one of the frontal joints 14.

In the figures, it can be seen that the medial rail 10 and the lateral rail 8 extend parallel to each other in each position, so that the angle between them does not change. This is achieved by the special shape of the slot 20, in that an orientation of the two accommodation elements (16,18) relative to each other also remains constant. The displacement of the two accommodation elements is collinear.

FIG. 9 depicts a part of a knee orthosis according to a further example of an embodiment of the present invention. Only the upper leg part 4 is shown. In each of the three representations in FIG. 9, the upper leg part 4 with a lateral rail 8 and a medial rail 10 is depicted, each of which comprise a frontal joint 14 via which the respective two components 12 are connected to each other. The first accommodation element 16 and the second accommodation element 18 can be seen between the components 12 depicted above the respective frontal joints 14. In contrast to the example of an embodiment shown thus far, both the first accommodation element 16 and the second accommodation element 18 are arranged on both the lateral rail 8 and the medial rail 10. The connections between two of these components are swivel connections, as can be clearly seen in the different representations in FIG. 9.

The first accommodation element 16 has a projection 26 that extends upwards, with which it engages in a locking element 28. It can be seen that, in the left-hand representation of FIG. 9, the projection 26 is arranged in the right-hand section of the locking device 28, while in the right-hand representation of FIG. 9 it is arranged in the left-hand section of the locking device and in the middle section of the locking device in the middle part of the representation. This changes not only the position of the first accommodation element 16 relative to the second accommodation element 18, but also the angle between the two components 12, which are connected by the two accommodation elements 16, 18, and thus also a frontal knee angle of the knee orthosis.

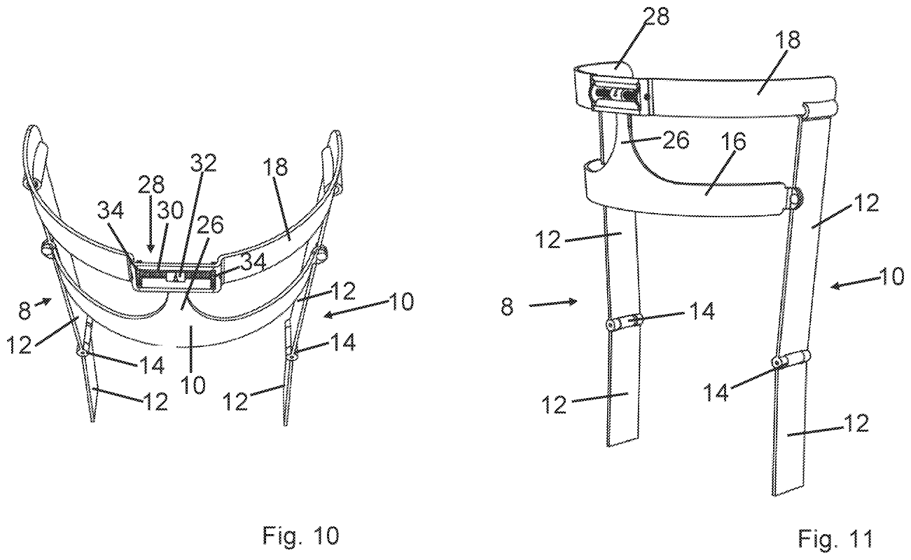

FIG. 10 depicts the part of the device from FIG. 10 in an oblique view from above. The first accommodation element 16 with the projection 26 as well as the second accommodation element 18 can be seen, both of which are connected to the components 12, respectively, which are connected to further components via frontal joints 14 and, together with said components, form the lateral rail 8 and the medial rail 10. The locking device 28 is shown in particular detail in this representation. It features a threaded rod 30 which interacts with an inner thread on a connecting piece 32 that is arranged on the projection 26. The threaded rod 30 can be rotated by way of two knurled wheels 32, whereby the connecting piece 32 and thus also the projection 26 and the first accommodation element 16 are displaced relative to the locking device 28 and thus also relative to the second accommodation element 18, whereby the three positions shown in FIG. 9 as well as all intermediate positions can be reached.

FIG. 11 shows the representation in FIG. 10 from a different perspective. Again, the components 12 can be seen which form the medial rail 10 and the lateral rail 8 and which are each connected to each other by a frontal joint 14. The first accommodation element 16 and the second accommodation element 18 are attached to the medial rail 10 and the lateral rail 8, respectively.

To ensure that the device can be swivelled into the position shown in FIG. 9, it is advantageous if the pivot points, at which the various components are connected to each other, are designed to be variable on at least one side, for example in the form of an elongated hole. This applies particularly for at least one attachment of the first accommodation element 16 to one of the components 12. Preferably, the locking device 28 or at least a part of it, for example the threaded rod 30, which is already designed to be rotatable, is configured on a side with a variable pivot point. It is advantageous if this is the same side on which the pivot point between the first accommodation element 16 and the respective component 12 is also designed as a variable pivot point.

### REFERENCE LIST

2 lower leg part

4 upper leg part

6 swivel joint

8 lateral rail

10 medial rail

12 component

14 frontal joint

16 first accommodation element

18 second accommodation element

20 slot

22 pin

24 hole

26 projection

28 locking device

30 threaded rod

32 connecting piece

34 knurled wheel

1. A knee orthosis with
a lower leg part that extends in a lower leg direction, and
an upper leg part that extends in a upper leg direction, and
at least one locking device,
wherein the lower leg part and the upper leg part are connected to each other by at least one swivel joint such that they can be swiveled,
the lower leg part and/or the upper leg part comprise a lateral rail and a medial rail, and
the lower leg direction encloses a knee frontal angle with the upper leg direction, characterized in that the knee orthosis has a first accommodation element and a second accommodation element which can be fixed in different positions with different knee frontal angles relative to one another by means of the at least one locking device.

2. The knee orthosis according to claim 1, wherein the first accommodation element is attached to the lateral rail and the second accommodation element to the medial rail.

3. The knee orthosis according to claim 2, wherein the first accommodation element and the second accommodation element each are attached to the lateral rail and the medial rail, preferably such that they can be swiveled.

4. The knee orthosis according to claim 1, wherein the first accommodation element and the second accommodation element each comprise at least one positive-locking element, which are designed to correspond with one another.

5. The knee orthosis according to claim 1, wherein at least one of the positive-locking elements is a slot, in which the other positive-locking element is mounted such that it can be moved, the slot preferably being designed in such a way that an orientation of the first accommodation element relative to the second accommodation element remains constant.

6. The knee orthosis according to claim 5, wherein the first accommodation element and/or the second accommodation element comprises multiple positive-locking elements arranged at an offset to each other.

7. The knee orthosis according to claim 1, wherein the at least one locking device is configured to fix the first accommodation element to the second accommodation element or vice-versa.

8. The knee orthosis according to claim 7, wherein the locking device is designed in the form of a velcro element, preferably arranged on one of the accommodation elements, and at least one velcro counter-element, preferably arranged on the other accommodation element.

9. The knee orthosis according to claim 1, wherein the lateral rail (8) and/or the medial rail (10) comprises at least two components (12), which are connected to each other by a frontal joint (14).

10. The knee orthosis according to claim 9, wherein the least one locking device is configured to fix the frontal joint (14).

11. The knee orthosis according to claim 9, wherein the frontal joint features an adjustment device with which an angular position of the frontal joint can be adjusted.

12. The knee orthosis according to claim 1, wherein the medial rail and/or the lateral rail are designed to be elastic, for example made from a metal, especially a spring steel.

13. The knee orthosis according to claim 1, wherein the medial rail and/or the lateral rail are designed such that they are not variable in length.

14. A knee orthosis comprising:
a lower leg part that extends in a lower leg direction;
an upper leg part that extends in a upper leg direction;
at least one locking device; and
a first accommodation element and a second accommodation element, each of the first and second accommodation elements comprising at least one positive-locking element;
wherein the lower leg part and the upper leg part are connected to each other by at least one swivel joint such that they can be swiveled, the lower leg part and/or the upper leg part comprising a lateral rail and a medial rail, and
wherein the lower leg direction encloses a knee frontal angle with the upper leg direction, and wherein the first accommodation element is attached to the lateral rail and the second accommodation element is attached to the medial rail, and wherein the first and second accommodation elements can be fixed in different positions with different knee frontal angles relative to one another by means of the at least one locking device.

15. The knee orthosis of claim 14, wherein the first accommodation element and the second accommodation element are attached to the lateral rail and the medial rail, respectively, in a manner such that they can be swiveled.

16. The knee orthosis according to claim 14, wherein the positive locking elements of the first and second accommodation elements are designed to correspond with each other.

17. The knee orthosis of claim 16, wherein at least one of the positive-locking elements is a slot, in which the other positive-locking element is mounted such that it can be moved, the slot preferably being designed in such a way that an orientation of the first accommodation element relative to the second accommodation element remains constant.

18. The knee orthosis according to claim 14, wherein the locking device is designed in the form of a velcro element arranged on one of the accommodation elements and at least one velcro counter-element arranged on the other accommodation element.

19. The knee orthosis according to claim 14 wherein the lateral rail and/or the medial rail comprises at least two components connected to each other by a frontal joint, and wherein the frontal joint includes an adjustment device to allow an angular position of the frontal joint to be adjusted.

20. A knee orthosis comprising:
a lower leg part that extends in a lower leg direction;
an upper leg part that extends in a upper leg direction;
at least one velcro locking device; and
a first accommodation element and a second accommodation element, each of the first and second accommodation elements comprising at least one positive-locking element, the positive-locking elements being designed to correspond with each other;
wherein the lower leg part and the upper leg part are connected to each other by at least one swivel joint such that they can be swiveled, the lower leg part and/or the upper leg part comprising a lateral rail and a medial rail, and
wherein the lower leg direction encloses a knee frontal angle with the upper leg direction, and wherein the first accommodation element is attached to the lateral rail and the second accommodation element is attached to the medial rail, and wherein the first and second accommodation elements can be fixed in different positions with different knee frontal angles relative to one another by means of the at least one locking device.
